(19) United States
(12) Patent Application Publication
(10) Pub. No.: US 2008/0108078 A1
(43) Pub. Date: May 8, 2008

(54) GENES ASSOCIATED WITH MIGRAINE

(76) Inventor: Stephanie Chissoe, Durham, NC (US)

Correspondence Address: GLAXOSMITHKLINE;Corporate Intellectual Property, P. O. Box 13398, Five Moore Drive, Research Triangle Park, NC, 27709-3398, US

(21) Appl. No.: 11/933,474

(22) Filed: Nov. 1, 2007

Related U.S. Application Data
(60) Provisional application No. 60/864,680, filed on Nov. 7, 2006.

(51) Int. Cl. C12Q 1/68

(52) U.S. Cl. 435/6

(57) ABSTRACT

A method of screening a small molecule compound for use in treating Migraine, comprising screening a test compound against a target selected from the group consisting of the gene products encoded by APOE, GNAL, NEDD4L, PDIP, TPCN1, TRPM8, ADRA1B, P2RX4, TAAR2, TAAR3, USP11, CHRNA5, RAB5A, DPP8, F2RL1, FZD5, PTGER1, SPI, ALOX5, CMTM8, DCBLD2, DPYS, IKBKB, OVCH1, PDE4DIP, PPM1G, PYY2, RYR1, BRD2, CAD, F2RL2, NCOA3, ADORA2B, BMX, CHRNA3/CHRNB4, F2R, GRIK5, ITGB4, MAPK10, NPEPL1, PTGIS, UCN2, or WASF1, where activity against said target indicates the test compound has potential use in treating Migraine.

### CROSS-REFERENCE TO RELATED APPLICATIONS

This application claims priority to U.S. provisional patent application No. 60/864,680 filed Nov. 7, 2006.

### FIELD OF THE INVENTION

The present invention relates to identification of genes that are associated with Migraine and to screening methods to identify chemical compounds that act on those targets for the treatment of Migraine or its associated pathologies.

### BACKGROUND OF THE INVENTION

The purpose of the present study was to identify genes coding for tractable targets that are associated with Migraine, to develop screening methods to identify compounds that act upon such targets, and to develop such compounds as medicines to treat Migraine and its associated pathologies.

Family and twin studies indicate that genetic factors are involved in the aetiology of migraine. Migraine is a common neurological disorder, affecting approximately 12% of the adult population in Western countries. Epidemiological studies have shown that migraine is more common in females with a male:female ratio of 1:2-3. Prevalence is also related to age, with the young and middle-aged being predominantly affected and over 90% of patients reporting their first attack before the age of 40.

Migraine is an episodic condition in which headaches recur at irregular intervals with attacks typically lasting approximately one day. Due to the complexity of disease definition, diagnosis is not straightforward and represents a challenge for general practitioners. Migraine is recognised as occurring most commonly in one of two forms under the International Headache Society (IHS) classification of headache—migraine without aura and migraine with aura. Patients do, however, experience attacks of either type which can contribute to confusion in diagnosis. Migraine without aura affects around 85% of the migraine population. Migraine with aura affects around 15% of the migraine population.

Based on their clinical presentation, primary headache disorders (i.e., those that are not symptomatic of an underlying pathology such as a brain tumour or intracerebral haemorrhage) are generally divided into three groups: migraine headaches, cluster headaches, and tension headaches. Recent research from Canada suggests that patients with IHS defined headache have three variations of migraine/headache including tension. The proportions are 30% of attacks as IHS defined migraine, 60% of attacks have some migrainous characteristics but do not strictly meet IHS criteria, and 10% of attacks are tension.

### SUMMARY OF THE INVENTION

A first aspect of the present invention is a method for screening small molecule compounds for use in treating Migraine, by screening a test compound against a target selected from the group consisting of gene products of the genes APOE, GNAL, NEDD4L, PDIP, TPCN1, TRPM8, ADRA1B, P2RX4, TAAR2, TAAR3, USP 11, CHRNA5, RAB5A, DPP8, F2RL1, FZD5, PTGER1, SPI, ALOX5, CMTM8, DCBLD2, DPYS, IKBKB, OVCH1, PDE4DIP, PPM1G, PYY2, RYR1, BRD2, CAD, F2RL2, NCOA3, ADORA2B, BMX, CHRNA3/CHRNB4, F2R, GRIK5, ITGB4, MAPK10, NPEPL1, PTGIS, UCN2, and WASF1. Activity against said target indicates the test compound has potential use in treating Migraine.

### DETAILED DESCRIPTION

The present inventors tested genes that encode for potential tractable targets to identify genes that are associated with the occurrence of Migraine and to provide methods for screening to identify compounds with potential therapeutic effects in Migraine. An assessment of Migraine data was carried out with a pooled data set of 763 Caucasian cases and 769 Caucasian controls collected from Griffiths University in Queensland, Australia. Allelic and genotypic frequencies for the 5,983 Single Nucleotide Polymorphisms (SNPs) in 1,745 genes were contrasted between the cases and controls. In addition, gene-based permutation analyses were performed to account for the variable number of SNPs per gene. On the basis of these analyses, 11 genes or loci were identified as being significantly associated with Migraine: APOE, GNAL, NEDD4L, PDIP, TPCN1, TRPM8, ADRA1B, P2RX4, TAAR2, TAAR3, USP11. These genes all have a gene-based permutation P≦0.005 in the pooled data set. Likewise, an additional 17 genes showed statistical significance in the pooled data set with a permutation P>0.005 but <0.01. These genes are CHRNA5, RAB5A, DPP8, F2RL, FZD5, PTGER1, SPI, ALOX5, CMTM8, DCBLD2, DPYS, IKBKB, OVCH1, PDE4DIP, PPM1G, PYY2, AND RYR1. A combined assessment analysis revealed 15 more statistically significant genes (BRD2, CAD, F2RL2, NCOA3, ADORA2B, BMX, CHRNA3/CHRNB4, F2R, GRIK5, ITGB4, MAPK10, NPEPL1, PTGIS, UCN2, and WASF1) when splitting the pooled data into two randomized subsets. The thresholds were established on a continuum with a permutation P≦0.05 in the pooled data set and a minimum permutation P<0.20 in both of the two split subsets.

As used, herein, a ‘tractable target’ or ‘druggable target’ is a biological molecule that is known to be responsive to manipulation by small molecule chemical compounds, e.g., can be activated or inhibited by small molecule chemical compounds. Classes of ‘tractable targets’ include, but are not limited to, 7-transmembrane receptors (7™ receptors), ion channels, nuclear receptors, kinases, proteases and integrins.

An aspect of the present invention is a method for screening small molecule compounds for use in treating Migraine, by screening a test compound against a target selected from the group consisting of proteins encoded by the genes APOE, GNAL, NEDD4L, PDIP, TPCN1, TRPM8, ADRA1B, P2RX4, TAAR2, TAAR3, USP1, CHRNA5, RAB5A, DPP8, F2RL1, FZD5, PTGER1, SPI, ALOX5, CMTM8, DCBLD2, DPYS, IKBKB, OVCH1, PDE4DIP, PPM1G, PYY2, RYR1, BRD2, CAD, F2RL2, NCOA3, ADORA2B, BMX, CHRNA3/CHRNB4, F2R, GRIK5, ITGB4, MAPK10, NPEPL1, PTGIS, UCN2, and WASF1. Activity against said target indicates the test compound has potential use in treating Migraine. Activity may be enhancing (increasing) the biological activity of the gene product, or diminishing (decreasing) the biological activity of the gene product.

### EXAMPLE 1

### Subjects and Methods

### Sample Set

The sample set consisted of 800 Caucasian cases and 800 unrelated Caucasian Controls of which 763 Caucasian cases and 769 Caucasian were used in the study. The subjects were collected from Griffiths University in Queensland, Australia with recruitment ending in July 2003. All Caucasian individuals recruited into this study were defined as being of British descent. All subjects gave informed consent for the use of their DNA in this study prior to recruitment.

All Migraine individuals were included in the study if they did not meet any of the exclusion criteria below. Migraine individuals could be included if they had a single life episode that resulted in depression for a short period of time less than 12 Months. Controls were excluded based on the exclusion factors below.

### Exclusion of Migraine Individuals (Cases)

An incorrectly signed and dated consent form and/or questionnaire
More than 6 glasses of alcohol a day prior to (pre) onset or after (post) onset of migraine.

Pre-Onset of Migraine:—
Subject has a history of chronic cerebrovascular pathology including stroke
Subject has a history of chronic depression or schizophrenia
Subject has a history of chronic epilepsy
Subject has had a head, neck or back injury or trauma
   Exclusion of Controls
   Controls must be excluded if they:

have ever suffered from a migraine.
have a first degree relative (parent, sibling or child) that is affected or has been affected with migraine.
drink more than 6 glasses of alcohol a day
have any of the following disorders prior to the age of 17.7 years in females and 21.2 years in males:
   history of chronic cerebrovascular pathology including stroke
   history of chronic depression or schizophrenia
   history of chronic epilepsy
   has had a head, neck or back injury or trauma
      Target Genes

Relatively few human proteins, approximately a hundred in total, are considered to be suitable targets for effective small molecule drugs. It was considered reasonable to include all the members of these families for which a sequence was available. At the time, some of the genes were not exemplified in the public domain and were discovered through the analysis of expressed sequence tags or genomic sequence using a combination of sequence analysis. In addition, genes were selected because they were the targets of effective drugs even though they were not part of large protein families. Finally, disease expertise was employed to select genes whose involvement in Migraine was either proven or suspected. Although over 2000 genes were selected in total, only 1,745 genes were analyzed was due to attrition in SNP identification, primer design, genotyping and data quality control. Genes were named accordingly to NCBI ENTREZ gene.

SNP Identification

The genes were automatically assembled and annotated with a region of the gene designated as 5′ and 3′, intron and exon. SNPs were mapped using BLAST to the manually curated genomic sequences. The SNPs were selected up to 10 kb from the start and stop sites of the transcripts with an average intermarker distance of 30 Kb. SNPs with a minor allele frequency (MAF)>5% were selected, but, all known coding SNPs were included irrespective of MAF. Approximately 10% of genes had fewer than 6 SNPs and these were subjected to SNP discovery using 24 primer pairs per gene to amplify 12 DNAs selected from Coriell Cell Repository of female CEPH cell-line samples. (CEPH refers to the Centre d'Etude du Polymorphisme Humain, which collected Northern European DNA samples.) For all of the discovered SNPs a minor allele frequency was determined using the FAST (Flow Accelerated SNP Typing) (Taylor et al, 2001) technology using multiplex PCR coupled with Single Base Chain Extension (SBCE) and Amplifluor genotyping. A marker selection algorithm was used to remove highly correlated SNPs to reduce the genotyping requirement while maintaining the genetic information content throughout the regions (Meng et al, 2003).

Sample Preparation and Genotyping

DNA was isolated from whole blood using a basic salting-out procedure. Samples were arrayed and normalized in water to a standard concentration of 5 ng/ul. Twenty nanogram aliquots of the DNA samples were arrayed into 96-well PCR plates. For purposes of quality control, 3.4% of the samples were duplicated on the plates and two negative template control wells received water. The samples were dried and the plates were stored at −20° C. until use. Genotyping was performed by a modification of the single base chain extension (SBCE) assay previously described (Taylor et al. 2001). Assays were designed by a GlaxoSmithKline in-house primer design program and then grouped into multiplexes of 50 reactions for PCR and SBCE. Following genotyping, the data was scored using a modification of Spotfire Decision Site Version 7.0 Genotypes passed quality control if: a) duplicate comparisons were concordant, b) negative template controls did not generate genotypes and c) more than 80% of the samples had valid genotypes. Genotypes for assays passing quality control tests were exported to an analysis database.

Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples and phenotypic data at all times. All subjects gave informed consent for the use of their DNA and phenotypic data in this study. The analytical tools used in the analysis process described below interface directly with subject data in SubjectLand. This interface also archives the files used in analysis as well as the results.

Analysis

Only subjects with a subject type (SBTY) of case or control were analyzed. Subjects with a SBTY of affected family member or other SBTY values were excluded from analysis. Subjects were also excluded if he/she, either parent, or more than one grandparent were non-Caucasian as indicated by self-report. In addition, subjects were excluded if their putative gender was inconsistent with SNP genotypes on the X chromosome. Finally, subjects that genotyped on fewer than 75% of the SNPs in a given genotyping experiment were excluded from analysis.

Each marker was examined for Hardy-Weinberg equilibrium and minor allele frequency. Genotypic and allelic associations test were then performed, followed by identification of the risk allele and risk genotype using chi-square tests. An odds ratio and confidence interval of greater than 95% was calculated for the risk allele and risk genotype. Next, population stratification was evaluated by determining if the number of allelic and genotypic tests observed to be significant at a given threshold was inflated with respect to what would be expected under the null hypothesis of no association. In addition, linkage disequilibrium (LD) was examined to measure the association between alleles at different loci (Weir, 1996, pp. 109-110). Lastly, a permutation assessment was conducted to account for the variable number of SNPs per gene and yield a single permutation p-value per gene for the pooled analysis data set. Statistically significant genes were identified as those passing gene-based permutation thresholds. The empirical permutation p-value from the pooled data set was required to fall at or below 0.005 to be considered significantly associated with Migraine. Further, since the weight of statistical evidence occurs on a continuum, genes with a p-value greater than 0.005 or less than or equal to 0.01 were also considered statistically significant.

A combined assessment was also conducted whereby subjects from the pooled data set were randomly assigned to one of two subsets in order to yield a pair of “split” data sets. This randomization was done to ensure that the two subsets were as homogeneous as possible. In each of the three data sets the (one pooled and two split sets), allelic and genotypic frequencies were contrasted between cases and controls followed by gene-based permutation analyses. Genes were considered statistically significant on a continuum with a permutation P≦0.05 in the pooled data set and a minimum permutation P<0.20 in both of the two split subsets.

Hardy Weinberg Equilibrium

Hardy Weinberg equilibrium (HWE) is a measure of the association between two alleles at an individual locus. A bi-allelic marker is in HWE if the genotype frequencies are p2, 2pq and q2 for the genotypes 1, 1; 1, 2; and 2, 2 where p and q are the frequencies of the 1 and 2 alleles, respectively. The departure from HWE was tested using a Chi square test, by testing the difference between the expected (calculated from the allele frequencies) and observed genotype frequencies. A HWE permutation test was performed when the HWE chi-square p-value <0.05 and when at least one genotype cell had an expected count less than 5 (Zaykin et al, 1995). When these conditions exist, the HWE chi-square test may not be valid and a permutation test to assess departure from HWE is warranted. Markers failing HWE at p≦0.001 in controls were removed from the pooled analysis marker cluster used in association analyses. HWE failure may indicate a non-robust assay.

Minor Allele Frequency

For minor allele frequency, markers which were monomorphic were removed from the analysis marker cluster used in association analyses.

Allelic and Genotypic Test of Association

Testing for association in the study data was carried out using the ‘PROC FREQ’ fast Fisher's exact test (FET) procedure in the statistical software package SASv8.2. An exact test is warranted in situations when asymptotic assumptions are not met such as when the sample size is not large or when the distribution is sparse or skewed. Such situations occur for SNPs with rare minor allele frequencies where the number of expected cases and/or controls for the rare homozygote are less than 5. Under these conditions, the asymptotic results many not be valid and the asymptotic p-value may differ substantially from the exact p-value. The classic Fisher's Exact Test computes exact p-values by enumerating all tables as extreme as, or more extreme than, that observed. This direct enumeration approach is very time-consuming and only feasible for small problems. The fast Fisher's Exact test computes exact p-values for general R×C tables using the network algorithm developed by Mehta and Patel (1983). The network algorithm provides substantial advantage over direct enumeration and is rapid and accurate.

Tables I and II show the structure of the genotype and allele contingency tables, respectively

TABLE I
Generic disease status by genotype contingency table.
Disease Status

| | | Case | Control | Total |
|---|---|---|---|---|
| Genotype | AA | n11 | n12 | n1. |
| | Aa | n21 | n22 | n2. |
| | aa | n31 | n32 | n3. |

| | | | |
|---|---|---|---|
| Total | n.1 | n.2 | N |

TABLE II
Generic disease status by allele contingency table.
Disease Status

| | | Case | Control | Total |
|---|---|---|---|---|
| Allele | A | 2n11 + n21 | 2n12 + n22 | 2n1. + n2. |
| | a | 2n31 + n21 | 2n32 + n22 | 2n3. + n2. |

| | | | |
|---|---|---|---|
| Total | 2n.1 | 2n.2 | 2N |

Risk Allele and Risk Genotype

The “risk allele” refers to the allele that appeared more frequently in cases than controls. The “risk genotype” was determined after identifying the genotype that had the largest chi-square value when compared against the other 2 genotypes combined in the genotypic association test. For example, if a SNP had genotypes AA, AG and GG, 3 chi-square tests were performed contrasting cases and controls: 1) AA vs AG+GG, 2) AG vs AA+GG and 3) GG vs AA+AG. An odds ratio was then calculated for the test with the largest chi-square statistic. If the odds ratio was >1, this genotype was reported as the risk genotype. If the odds ratio was <1, then 1) the risk genotype was reported as “!” (“!” means “not”) this genotype and 2) a new odds ratio was calculated as the inverse of the original odds ratio. This new odds ratio was reported.

Odds Ratios and Confidence Intervals

An odds ratio was constructed for the risk allele and risk genotype.

Odds ratio (OR)=(n11*n22)/(n12*n21)
where
   n11=cases with risk genotype
   n21=cases without risk genotype
   n12=controls with risk genotype
   n22=controls without risk genotype
In order to avoid division or multiplication by zero, 0.5 was added to each cell in the contingency table (as recommended in “Statistical Methods for Rates and Proportions” by Fleiss, Ch 5.3 p. 64)
A 95% confidence interval for the odds ratio was also calculated as follows: where
   z=97.5th percentile of the standard normal distribution
   v=[1/(n11)]+[1/(n12)]+[1/(n21)]+[1/(n22)]
      Evaluation of Population Stratification

In this assessment, cases and control frequencies were compared across a subset of relatively independent markers (markers in low LD) selected from the set of all markers analyzed. Since the vast majority of genes on the gene list are not associated with a specific disease, this constitutes a null data set. If the cases and controls are from the same underlying population, the expectation is to see 5% of the tests significant at the 5% level, 1% significant at the 1% level, etc. If, on the other hand, the cases and controls are from different populations, (for example, cases from Finland and controls from Japan), there would be an inflation in the proportion of tests significant across thresholds due to genetic differences between the two populations that are unrelated to disease. Inflation in the number of observed significant tests over a range of cut-points suggests that the case and control groups are not well matched. Consequently, the inflated number of positive tests may be due to population stratification rather than to association between the associated SNPs and disease.

The probability of ≧m observed number of significant tests out of n total tests at a cut-point p was calculated using the binomial probability as implemented in either S-PLUS or SAS.

With SAS PROBNML (p,n,m) computes the probability that an observation from a binomial (n,p) distribution will be less than or equal to m.

Linkage Disequilibrium

The LD between two markers is given by DAB=pAB−pApB, where pA is the allele frequency of A allele of the first marker, pB is the allele frequency of B allele of the second marker, and pAB is the joint frequency of alleles A and B on the same haplotype. LD tends to decline with distance between markers and generally exists for markers that are less than 100 kb apart

The SAS procedure PROC CORR was used to calculate r using the Pearson product-moment correlation. To determine whether significant LD existed between a pair of markers we made use of the fact that nr2 has an approximate chi square distribution with 1 df for biallelic markers. The significance level of pairwise LD was computed in SAS.

Permutation Assessment

The analysis of the observed un-permuted data led to a set of observed p-values for each gene. We defined min [obs(p)] as the minimum p-value derived from all tests of all SNPs within the gene for a given data set. The objective of this permutation test was to determine the significance of this minimum p-value in context of the number of SNPs analyzed number of tests conducted and the correlation between SNPs within each gene. The permutation process accounted for the multiple SNPs and tests conducted within a particular gene but it did not account for the total number of genes being analyzed.

Due to computational limitations, only those genes with a min [obs (p)] less than a threshold of 0.05 were assessed for significance using a permutation process. A maximum number of permutations, N, was conducted per gene (N=50,000 for pooled set; see below). However, this maximum number did not need to be conducted for every gene. For many genes far fewer permutations were sufficient to show that a gene was not significant at the threshold of interest and the permutation process for that gene was terminated early.

The following process was followed. For each permutation, affection status was shuffled among the cases and controls, maintaining the overall number of cases and number of controls in the observed data. The genetic data for each subject were not altered. For each permutation, all the SNPs within a gene were analyzed using allelic and genotypic association tests (same methods as employed with true, observed data). The p-value for the most significant test, min [sim (p)] was captured for each permutation. The permutations were repeated up to N times such that up to N min [sim (p)]'s were captured. Once the permutations were completed, the min [obs (p)] for each gene was compared against the distribution of min [sim (p)]. The proportion of min [sim (p)] that was less than the min [obs (p)] gave the empirical permutation p-value for that gene. This p-value was labelled perm (p).

The maximum number of iterations needed to accurately assess the permutation p-value depended on the threshold set for declaring significance. For example, in assessing permutation p-values below 0.05, 5000 permutations gave a 95% confidence interval (CI) of 0.044 to 0.056. This was not considered to be a tight enough estimate of the true permutation p-value. By assessing 50,000 permutations the 95% CI was narrowed considerably, to 0.48 to 0.52. The CIs for a range of permutation p-values and numbers of permutations are presented below.

| permP | 5000 CI | 10000 CI | 50000 CI |
|---|---|---|---|
| 0.05 | (0.044, 0.056) | (0.0457, 0.0543) | (0.048, 0.052) |
| 0.01 | (0.0072, 0.0128) | (0.008, 0.012) | (0.0091, 0.011) |
| 0.005 | (0.003, 0.008) | (0.0036, 0.0064) | (0.0044, 0.0056) |

Based on the above CI estimates, genes in the pooled data set with an obs (p)≦0.05 were assessed with a maximum of 50,000 permutations.

### EXAMPLE 2

### Results

Thirty-two collected subjects were excluded from the study based on sample set quality control (QC) measures. Five for ethnicity, 18 for gender inconsistency, and 9 that genotyped on fewer than 75% of the SNPs. The mean age at recruitment and proportion of males for cases and controls was similar. Key demographic characteristics of the pooled data set are detailed in Table 1.

During SNP marker quality control, 59 SNPs were excluded due to Hardy-Weinberg Equilibrium (HWE); 270 SNPs were excluded because SNPs were monomorphic in cases and controls; 21 SNPs were excluded due to mapping issues. As a result, 5,983 SNPs were analyzed for association with Migraine of which 5,913 had a gene assignment and 70 did not. In total 1,745 genes were analyzed: 1,680 autosomal, 65 X-linked. The mean number of SNPs per genes was 3.4 with a range of 1-53 SNPs per gene. See Table 2 for a summary SNP coverage of genes.

Detailed summaries of genotype counts across all genes and subjects analysed are given in Table 3 and Table 4. The apparent bimodal distribution seen in the tables reflect the staged genotyping process and the evolution of the gene list over time.

After gene-based permutation analysis, 11 genes were identified as having the strongest statistical evidence for genetic associated with Migraine (Table 5). The set of genes reached a gene-based permutation P-value of <=0.005 in the pooled data set of all 763 cases and 769 controls. The 17 genes in Table 6 are the next best in terms of statistical evidence. These genes have a gene-based permutation P-value between 0.005 and 0.01.

The number of tests significant across various thresholds was not inflated beyond what is expected by chance (Table 7).

Using a combined assessment of pooled and split subsets, genes in Table 8 showed statistical evidence at permutation P≦0.05 in the pooled data set and a minimum permutation P<0.20 in both of the two split subsets. Given that there is significant overlap with these results and those identified by the pooled only approach, only 15 new genes were identified using this statistical method.

APOE, GNAL, NEDD4L, PDIP, TPCN1, TRPM8, ADRA1B, P2RX4, TAAR2, TAAR3, USP11, CHRNA5, RAB5A, DPP8, F2RL, FZD5, PTGER1, SPI, ALOX5, CMTM8, DCBLD2, DPYS, IKBKB, OVCH1, PDE4DIP, PPM1G, PYY2, AND RYR1 passed statistically significant gene-based permutation thresholds in the pooled data set. These genes have the strongest statistical evidence for association with Migraine. Further, there was no evidence of population stratification based on the distribution of results.

However, it is possible that some of the associations are false positives. Statistical association between a polymorphic marker and disease may occur for several reasons. The marker may be a mutation that influences disease susceptibility directly or may be correlated with a mutation that influences disease susceptibility because the marker and disease susceptibility mutation are physically close to one another. Spurious association may result from issues such as confounding or bias although the study design attempts to remove or minimize these factors. The association between a marker and disease may also be due to chance.

The gene-wise type 1 error is the gene-based permutation p-value threshold used to identify the genes of interest. It also provides the false positive rate associated with each gene. Out of 1,745 genes examine, an average of 8.7±2.9 would be expected to have a permutation p≦0.005 while 17.5±4.2 would be expected to have a permutation p≦0.01.

For the combined assessment, BRD2, CAD, F2RL2, NCOA3, ADORA2B, BMX, CHRNA3/CHRNB4, F2R, GRIK5, ITGB4, MAPK10, NPEPL1, PTGIS, UCN2, and WASF1 passed statistically significant gene-based permutation thresholds in the pooled data set and split subsets.

TABLE 1
Collections analysed:

| | Cases | Controls |
|---|---|---|
| Case/control status - total | | |
| Pooled Data Set | 763 | 769 |
| Migraine with Aura by IHS¹ | | |
| Pooled Data Set | 608 | 0 |
| Migraine without Aura by IHS¹ | | |
| Pooled Data Set | 148 | 0 |
| Migraine with Aura not by IHS² | | |
| Pooled Data Set | 6 | 0 |
| Migraine without Aura not by IHS² | | |
| Pooled Data Set | 1 | 0 |
| Male:Female | | |
| Pooled Data Set | 130:633 | 132:637 |
| Mean Age at Onset/Age at Exam | | |
| Footed Data Set | 49.03 | 50.82 |

¹Migraine classification meets strict IHS (International Headache Society) criteria
²Migraine classification does not meet strict IHS criteria

TABLE 2
SNP coverage of genes in analysis marker cluster

| | 1 | 2 | 3 | 4-5 | 6-9 | 10+ | |
|---|---|---|---|---|---|---|---|
| | SNP | SNPs | SNPS | SNPs | SNPs | SNPs | Total |
| No. genes | 448 | 480 | 340 | 230 | 153 | 94 | 1,745 |

TABLE 3
Summary of genotype counts across SNPs

| Numbers of genotypes | Number of markers |
|---|---|
| 1401-1532 | 4,106 |
| 1201-1400 | 129 |
| 1001-1200 | 2 |
| 801-1000* | 403 |
| <801* | 1,343 |

TABLE 4
Summary of genotype counts across subjects

| Numbers of genotypes | Number of subjects |
|---|---|
| 5501-5,983 | 77 |
| 5001-5500 | 457 |
| 4501-5000 | 617 |
| 4001-4500 | 363 |
| 3501-4000 | 11 |
| <3501 | 7 |

TABLE 5
Genes with Permutation P-value ≦ 0.005 in pooled set

| | Permutation | | |
|---|---|---|---|
| REGION | P | Target Class | Description |
| APOE | 0.0024 | OTHER_TARGETS | apolipoprotein E |
| GNAL | 0.0023 | Unclassified | guanine nucleotide binding |
| | | | protein (G protein), alpha |
| | | | activating activity polypeptide, |
| | | | olfactory type |
| NEDD4L | 0.0002 | NR_COFACTOR | neural precursor cell |
| | | | expressed, developmentally |
| | | | down-regulated 4-like |
| PDIP | 0.0047 | Unclassified | axin 1 |
| PDIP | | Unclassified | protein disulfide isomerase, |
| | | | pancreatic |
| TPCN1 | 0.0047 | ION_CHANNEL | “two-pore channel 1, |
| | | | homolog” |
| TRPM8 | 0.0008 | ION_CHANNEL | transient receptor potential |
| | | | cation channel, subfamily M, |
| | | | member 8 |
| ADRA1B | 0.0018 | 7TM | adrenergic, alpha-1B-, |
| | | | receptor |
| P2RX4 | 0.0007 | ION_CHANNEL | purinergic receptor P2X, |
| | | | ligand-gated ion channel, 4 |
| GPR58 (aka TAAR3) | 0.0014 | 7TM | G protein-coupled receptor 58 |
| GPR57 (aka TAAR2) | 0.0014 | 7TM | G protein-coupled receptor 57 |
| USP11 | 0.0020 | PROTEASE | ubiquitin specific protease 11 |

TABLE 6
Genes with Permutation P-value between 0.005 and 0.01 in Pooled set

| | Permutation | | |
|---|---|---|---|
| REGION | P | Target Class | Description |
| CHRNA5 | 0.0077 | ION_CHANNEL | cholinergic receptor, nicotinic, alpha |
| | | | polypeptide 5 |
| DPP8 | 0.0094 | PROTEASE | dipeptidylpeptidase 8 |
| F2RL1 | 0.0053 | 7TM | coagulation factor II (thrombin) |
| | | | receptor-like 1 |
| FZD5 | 0.0078 | 7TM | frizzled homolog 5 (Drosophila) |
| PTGER1 | 0.0087 | 7TM | prostaglandin E receptor 1 (subtype |
| | | | EP1) |
| RAB5A | 0.0084 | UNCLASSIFIED | RAB5A, member RAS oncogene |
| | | | family |
| SP1 | 0.0077 | UNCLASSIFIED | Sp1 transcription factor |
| ALOX5 | 0.0062 | OTHER_ENZYMES | arachidonate 5-lipoxygenase |
| CKLFSF8 (aka CMTM8) | 0.0060 | ION_CHANNEL | chemokine-like factor super family 8 |
| DPYS | 0.0087 | PROTEASE | dihydropyrimidinase |
| ESDN (aka DCBLD2) | 0.0093 | Unclassified | endothelial and smooth muscle cell- |
| | | | derived neuropilin-like protein |
| IKBKB | 0.0072 | KINASE | inhibitor of kappa light polypeptide |
| | | | gene enhancer in B-cells, kinase |
| | | | beta |
| OVCH1 | 0.0058 | PROTEASE | similar to polyprotein - African |
| | | | clawed frog |
| PDE4DIP | 0.0051 | OTHER_TARGETS | phosphodiesterase 4D interacting |
| | | | protein (myomegalin) |
| PPM1G | 0.0052 | OTHER_ENZYMES | protein phosphatase 1G (formerly |
| | | | 2C), magnesium-dependent, |
| | | | gamma isoform |
| PYY2 | 0.0067 | 7TM_LIGAND | peptide YY, 2 (seminalplasmin) |
| RYR1 | 0.0085 | ION_CHANNEL | ryanodine receptor 1 (skeletal) |

TABLE 7
Assessment of Population Stratification

| | Total No. | | | | |
|---|---|---|---|---|---|
| | genotypic | Genotypic Association | | Allelic Association | |
| Observed p- | or allelic | No. tests < | Binomial | No. tests < | Binomial |
| values = p | tests | p(m) | prob ≧ m | p(m) | prob ≧ m |
| P < 0.05 | 2,174 | 115 | 0.24910 | 120 | 0.12371 |
| P < 0.01 | 2,174 | 20 | 0.59255 | 30 | 0.03489 |
| P < 0.005 | 2,174 | 8 | 0.75693 | 15 | 0.08531 |
| P < 0.001 | 2,174 | 1 | 0.63919 | 2 | 0.37033 |
| P < 0.0005 | 2,174 | 1 | 0.29622 | 1 | 0.29622 |

TABLE 8
Combined Assessment Significan Genes¹

| | Permutation P-value | | | | | |
|---|---|---|---|---|---|---|
| | Split | Split | Pooled | | Target | |
| Region² | subset 1 | subset 2 | set³ | Gene | Class | Gene Description |

Permutation P < 0.05 in pooled set and <0.05 in both split subsets.
Gene-wise type 1 error rate = 0.00125

| | | | | | | |
|---|---|---|---|---|---|---|
| NEDD4L | 0.0485 | 0.0079 | 0.0002 | NEDD4L | NR | neural precursor cell expressed, |
| | | | | | COFACTOR | developmentally down-regulated 4-like |
| TPCN1 | 0.0166 | 0.0178 | 0.0047 | TPCN1 | ION CHANNEL | “two-pore channel 1, homolog” |
| TRPM8 | 0.0347 | 0.0395 | 0.0008 | TRPM8 | ION CHANNEL | transient receptor potential cation channel, |
| | | | | | | subfamily M, member 8 |

Permutation P < 0.05 in pooled set and <0.10 in both split subsets.
Gene-wise type 1 error rate = 0.00434

| | | | | | | |
|---|---|---|---|---|---|---|
| BRD2 | 0.0107 | 0.0866 | 0.0126 | BRD2 | KINASE | bromodomain containing 2 |
| CAD⁴ | 0.0736 | 0.0816 | 0.0101 | SLC5A6 | TRANSPORTER | solute carrier family 5 (sodium-dependent |
| | | | | | | vitamin transporter), member 6 |
| | | | | CAD | PROTEASE | carbamoyl-phosphate synthetase 2, aspartate |
| | | | | | | transcarbamylase, and dihydroorotase |
| | | | | SLC30A3 | TRANSPORTER | solute carrier family 30 (zinc transporter), |
| | | | | | | member 3 |
| CHRNA5 | 0.0070 | 0.0584 | 0.0077 | CHRNA5 | ION CHANNEL | cholinergic receptor, nicotinic, alpha |
| | | | | | | polypeptide 5 |
| F2RL2 | 0.0337 | 0.0569 | 0.0151 | F2RL2 | 7TM | coagulation factor II (thrombin) receptor-like 2 |
| NCOA3 | 0.0910 | 0.0369 | 0.0166 | NCOA3 | NR COFACTOR | nuclear receptor coactivator 3 |
| RAB5A | 0.0546 | 0.0907 | 0.0084 | RAB5A | Unclassified | RAB5A, member RAS oncogene family |

Permutation P < 0.05 in pooled set and <0.15 in both split subsets.
Gene-wise type 1 error rate = 0.00871

| | | | | | | |
|---|---|---|---|---|---|---|
| ADORA2B | 0.1006 | 0.1072 | 0.0115 | ADORA2B | 7TM | adenosine A2b receptor |
| APOE | 0.0610 | 0.1277 | 0.0024 | APOE | OTHER | apolipoprotein E |
| | | | | | TARGETS | |
| CHRNA3_{—} | 0.0140 | 0.1425 | 0.0443 | CHRNA3 | ION CHANNEL | Cholinergic receptor, nicotinic, alpha |
| CHRNB4⁴ | | | | | | polypeptide 3 |
| | | | | CHRNB4 | ION CHANNEL | Cholinergic receptor, nicotinic, beta |
| | | | | | | polypeptide 4 |
| DPP8 | 0.0176 | 0.1327 | 0.0094 | DPP8 | PROTEASE | dipeptidylpeptidase 8 |
| FZD5 | 0.1035 | 0.0425 | 0.0078 | FZD5 | 7TM | frizzled homolog 5 (Drosophila) |
| GNAL | 0.0644 | 0.1359 | 0.0023 | GNAL | Unclassified | guanine nucleotide binding protein (G |
| | | | | | | protein), alpha activating activity |
| | | | | | | polypeptide, olfactory type |
| ITGB4 | 0.0308 | 0.1186 | 0.0196 | ITGB4 | INTEGRIN | integrin, beta 4 |
| PDIP | 0.1266 | 0.0348 | 0.0047 | AXIN1 | Unclassified | axin 1 |
| | | | | PDIA2 | Unclassified | protein disulfide isomerase, pancreatic |
| PTGIS | 0.1304 | 0.0622 | 0.0171 | PTGIS | Unclassified | prostaglandin I2 (prostacyclin) synthase |
| SP1 | 0.1260 | 0.0413 | 0.0077 | SP1 | Unclassified | Sp1 transcription factor |
| UCN2 | 0.0501 | 0.1296 | 0.0494 | UCN2 | 7TM LIGAND | stresscopin-related peptide |

Permutation P < 0.05 in pooled set and <0.20 in both split subsets.
Gene-wise type 1 error rate = 0.0139

| | | | | | | |
|---|---|---|---|---|---|---|
| BMX | 0.0410 | 0.1743 | 0.0129 | BMX | KINASE | BMX non-receptor tyrosine kinase |
| | | | | ACE2 | PROTEASE | angiotensin I converting enzyme (peptidyl- |
| | | | | | | dipeptidase A) 2 |
| F2R⁴ | 0.1516 | 0.0480 | 0.0240 | IQGAP2 | Unclassified | IQ motif containing GTPase activating |
| | | | | | | protein 2 |
| | | | | F2R | 7TM | coagulation factor II (thrombin) receptor |
| F2RL1 | 0.0149 | 0.1686 | 0.0053 | F2RL1 | 7TM | coagulation factor II (thrombin) receptor-like 1 |
| GRIK5 | 0.1838 | 0.0827 | 0.0216 | GRIK5 | ION CHANNEL | glutamate receptor, ionotropic, kainate 5 |
| MAPK10 | 0.1762 | 0.1719 | 0.0430 | MAPK10 | KINASE | mitogen-activated protein kinase 10 |
| NPEPL1 | 0.1709 | 0.0712 | 0.0182 | NPEPL1 | PROTEASE | aminopeptidase-like 1 |
| PTGER1⁴ | 0.1774 | 0.0053 | 0.0087 | RGS19IP1 | Unclassified | regulator of G-protein signalling 19 |
| | | | | | | interacting protein 1 |
| | | | | PTGER1 | 7TM | prostaglandin E receptor 1 (subtype EP1), |
| | | | | | | 42 kDa |
| | | | | PKN1 | KINASE | protein kinase C-like 1 |
| WASF1 | 0.0520 | 0.1673 | 0.0136 | WASF1 | Unclassified | WAS protein family, member 1 |

¹Accredited genes represent the set of genes that have passed a combined assessment of the primary and secondary screen data sets defined by T_{P} = 0.05 & T_{S} = 0.1.
²Region is a label used to assign a 1:1 relationship between a SNP and a unique part of the genome. In most instances the region and gene are one in the same. However, in gene rich parts of the genome (where SNPs map to multiple genes), a region may include several genes.
³The pooled set represents all 763 cases and 769 controls. The split subsets are the two randomised subsets selected from the pooled set.
⁴Some regions, in gene rich parts of the genome, have SNPs which map to several genes or have overlapping genes. The disease association may to be any one of these genes.

### REFERENCES

Fleiss J, Levin B., Paik M C. (2003) Statistical Methods for Rates and Proportions. 3rd Edition. John Wiley & Sons. Hoboken, N.J. Chapter 10, Pgs 234-283.
Mehta, C. and Patel, N. (1983) A Network Algorithm for Performing Fisher's Exact Test in rXc contingency tables. Journal of the American Statistical Association 78:427-434.
Meng, Z. et al. (2003) Selection of Genetic Markers for Association Analyses, Using Linkage Disequilbrium and Haplotypes. American Journal of Human Genetics 71(1): 115-130.
Roses A D., Burns D K., Chissoe S., Middleton L., St Jean P., (2005) Disease-specific target selection: A Critical First Step Down the Right Road. Drug Discovery Today 10: 177-189.
Taylor J D., Briley D., Nguyen Q., Long K., Tannone M A., Li M S., Ye F., Afshari A., Lai E., Wagner M., Chen J., Weiner M P. (2001) Flow cytometric platform for high-throughput single nucleotide polymorphism analysis. [Journal Article] Biotechniques. 30(3):661-6, 668-9, Mar.
Weir, B S. (1996) Genetic Data Analysis II. Sinauer Associates, Inc., Sunderland, Mass., pp. 109-110.
Zaykin D V, Zhivotovsky L A, Weir B S (1995) Exact tests for association between alleles at arbitrary numbers of loci. Genetica 96:169-178.

1. A method of screening a small molecule compound for use in treating Migraine, comprising screening a test compound against a target selected from the group consisting of the gene products encoded by APOE, GNAL, NEDD4L, PDIP, TPCN1, TRPM8, ADRA1B, P2RX4, TAAR2, TAAR3, USP11, CHRNA5, RAB5A, DPP8, F2RL1, FZD5, PTGER1, SPI, ALOX5, CMTM8, DCBLD2, DPYS, IKBKB, OVCH1, PDE4D1P, PPM1G, PYY2, RYR1, BRD2, CAD, F2RL2, NCOA3, ADORA2B, BMX, CHRNA3/CHRNB4, F2R, GRIK5, ITGB4, MAPK10, NPEPL1, PTGIS, UCN2, or WASF1, where activity against said target indicates the test compound has potential use in treating Migraine.
